(19) United States
(12) Patent Application Publication
(10) Pub. No.: US 2006/0287633 A1
(43) Pub. Date: Dec. 21, 2006

(54) Portable device for transfusing medical fluid

(76) Inventor: Jen-Hsien Yo, Taipei City (TW)

Correspondence Address: BACON & THOMAS, PLLC, 625 SLATERS LANE, FOURTH FLOOR, ALEXANDRIA, VA, 22314, US

(21) Appl. No.: 11/362,077

(22) Filed: Feb. 27, 2006

(30) Foreign Application Priority Data
Jun. 15, 2005 (TW) 94119881

(51) Int. Cl. A61M 1/00

(52) U.S. Cl. 604/317

(57) ABSTRACT

A portable device for transfusing medical fluid accommodated in a collapsible bag is provided. The portable device for transfusing medical fluid bagged in a collapsible bag comprises a container which includes a box-shaped body having a loading port for loading the bag of medical fluid and at least one fluid exit port, and a cove operably coupled to the box-shaped body for selective open/close the load port; a push plate movably disposed in the container and able to be displaced between a first position and a second position, wherein when the push plate is at the first position a space is defined within the container for receiving the bag of medical fluid and when the push plate is at the second position the space is reduced to a degree that the medical fluid in the collapsible bag is substantially discharged; a energy providing unit operably coupled to the push plate for providing energy required from displacing the push plate; and a lock/unluck means mounted to the container for operably locking the push plate at the first position or release the push plate to allow it to move to the second position.

### FIELD OF THE INVENTION

The present invention relates generally to a medical device and more particularly, to a portable device for transfusing medical fluid.

### BACKGROUND OF THE INVENTION

When people are sick and need to be treated with medicine, intravenous injection is one of the most commonly used way to allow medicine entering human body and for some medical treatment intravenous injection is the only way, such as hemodialysis. While performing intravenous injecting, in addition to insert a needle into a vein, placing a bag of medical fluid at a position higher than the place where the needle is inserted into the vein, such as hanging the bag/bottle containing medical fluid high on a post, to allow medical fluid to enter the vein by the effect of gravity is another important procedure to make this intravenous injection successful. During intravenous injecting, the bag/bottle containing the medical fluid has to be placed at a position higher than the place where the needle is inserted into the vein at all time so as to successfully transfusing the medical fluid into the human body. However, this will cause great inconvenience to patients who have the strength to move around, such as patients who have the strength to go for a walk or go to the bathroom.

Same situation happens to patients who utilize peritoneum to perform hemodialysis. A bag for containing the dialysis fluid is placed in the abdominal cavity of a patient who utilizes peritoneum to perform hemodialysis to allow metabolic waste products or toxic substances in the bloodstream to be exchanged into the dialysis fluid by way of the peritoneum. Then, after a period of time (normally 3 to 4 hours, depends on the volume of the dialysis fluid) the dialysis fluid will be saturated with the metabolic waste products or toxic substances and need to be discharged out of the abdominal cavity through a discharge port left on the belly of the patient. After the used dialysis fluid being discharged, fresh dialysis fluid then can be re-charged into the bag that is placed in the abdominal cavity of the patient to complete a hemodialysis treatment procedure. The advantage of this type of hemodialysis treatment is that the patient does not have to go to the hospital/clinic every 2 to 3 days to take the hemodialysis treatment. This type of hemodialysis treatment makes a long-term journey, such as overseas travel, possible and much easier. However, the space that the abdominal cavity can provide for accommodating the dialysis fluid is limited, thus the dialysis fluid need to be replaced every 3 to 4 hours. While recharging fresh dialysis fluid, a container containing fresh dialysis fluid has to be hanged or placed at a position higher than the patient's belly to charge the fresh dialysis fluid into the abdominal cavity by gravity. This charging procedure usually takes around 20 minutes to complete. Thus, for patient who is in a journey and is unable to fine a place to hang or place the bag that contains the dialysis fluid (such as a patient who is in a car or on an airplane), this dialysis fluid charging procedure causes a great deal of inconvenience.

In view of the inconvenience drawback in traditional intravenous injection and hemodialysis utilizing peritoneum, the inventor of the present invention has accomplished, after countless study and experiments, a portable device for transfusing medical fluid which can overcome the drawbacks of the traditional intravenous injection and hemodialysis utilizing peritoneum mentioned above and allow the patient to perform the traditional intravenous injection and hemodialysis utilizing peritoneum at anytime and anywhere he wants to.

### SUMMARY OF THE INVENTION

Accordingly, the object of the present invention is to provide a portable device for transfusing medical fluid which can effectively and speedily perform intravenous injection and dialysis fluid recharging procedure of a hemodialysis utilizing peritoneum at anytime and any where and without the limitation of the position of the container containing the medical fluid/dialysis fluid, so as to improve the quality of life of the patient.

To achieve this and other objects, a portable device for transfusing medical fluid accommodated in a collapsible bag is provided. The portable device for transfusing medical fluid bagged in a collapsible bag comprises a container which includes a box-shaped body having a loading port for loading the bag of medical fluid and at least one fluid exit port, and a cove operably coupled to the box-shaped body for selective open/close the load port; a push plate movably disposed in the container and able to be displaced between a first position and a second position, wherein when the push plate is at the first position a space is defined within the container for receiving the bag of medical fluid and when the push plate is at the second position the space is reduced to a degree that the medical fluid in the collapsible bag is substantially discharged; a energy providing unit operably coupled to the push plate for providing energy required from displacing the push plate; and a lock/unlock means mounted to the container for operably locking the push plate at the first position or release the push plate to allow it to move to the second position.

According to a preferred embodiment of the present application, the energy providing unit comprises a set of springs which are consisted of at lest one spiral springs, and the box-shaped body further comprises a stopping means for stopping the push plate at the second position.

It is to be appreciated that both the forging general description and the following detailed description are exemplary and explanatory and are intended to provide further explanations of the invention as claimed.

### BRIEF DESCRIPTION OF THE DRAWINGS

The accompanying drawings, which are included to provide a further understanding of the invention and are incorporated in and constitute a part of the specification, illustrate embodiments of the invention and together with the description serve to explain the principle of the invention. In the drawings,

FIG. 1 is an exploded perspective view of a portable device for transfusing medical fluid accommodated in a collapsible bag according to one embodiment of the present invention;

FIG. 2 is a perspective view of the portable device for transfusing medical fluid accommodated in a collapsible bag shown in FIG. 1;

FIG. 3a is a top perspective view of the push plate of the portable device for transfusing medical fluid accommodated in a collapsible bag shown in FIG. 1;

FIG. 3b is a bottom plane view of the push plate shown in FIG. 3a;

FIG. 4 is a perspective view of the portable device for transfusing medical fluid accommodated in a collapsible bag of the present invention showing the pushing spring being installed in the box-shaped body;

FIG. 5 is a perspective view of the portable device for transfusing medical fluid accommodated in a collapsible bag of the present invention showing that the push plate is locked at the first position by the lock member;

FIG. 6 is a perspective view of the portable device for transfusing medical fluid accommodated in a collapsible bag of the present invention showing that the push plate is pushed to the second position by the pushing springs;

FIG. 7 is a perspective view of the portable device for transfusing medical fluid accommodated in a collapsible bag of the present invention showing the use state of the portable device of the present invention.

### DETAILED DESCRIPTION OF THE INVENTION

Referring now to the drawings in which an embodiment of the present invention is illustrated to describe the present invention.

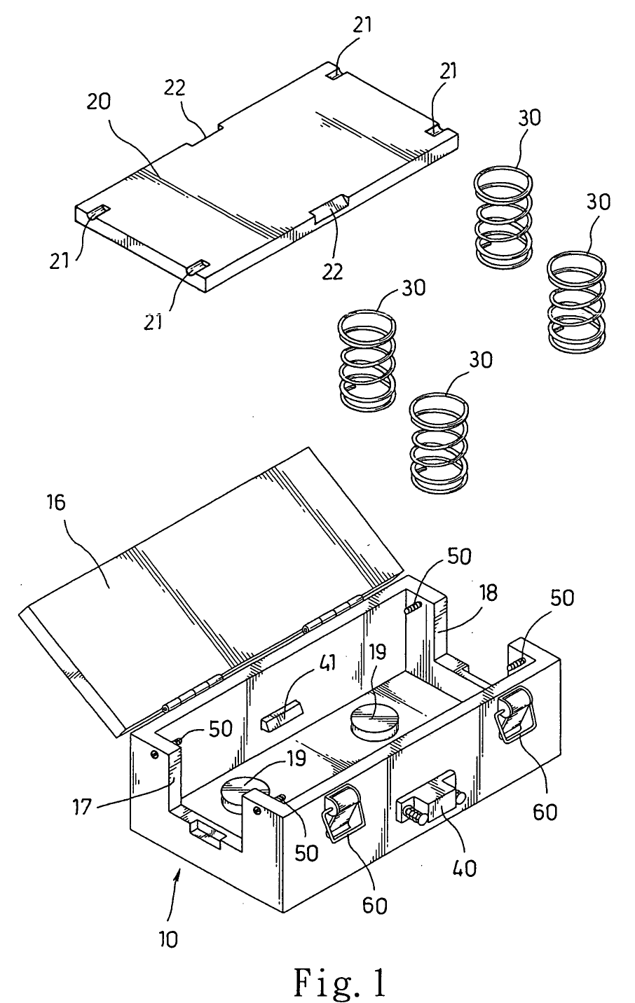

FIG. 1 is a perspective view of a portable device for transfusing medical fluid accommodated in a collapsible bag according to one embodiment of the present invention. As shown in FIG. 1, the portable device for transfusing medical fluid accommodated in a collapsible bag 1 generally comprises a container 1, a push plate 20, and a set of pushing springs 30 serving as an energy providing unit. In this embodiment, the set of pushing springs consist of four spiral springs 30.

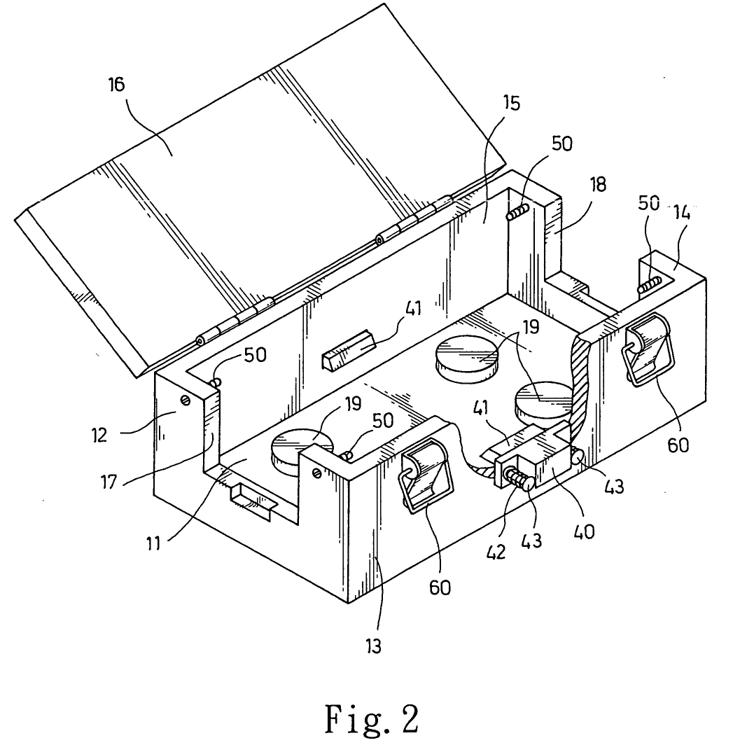

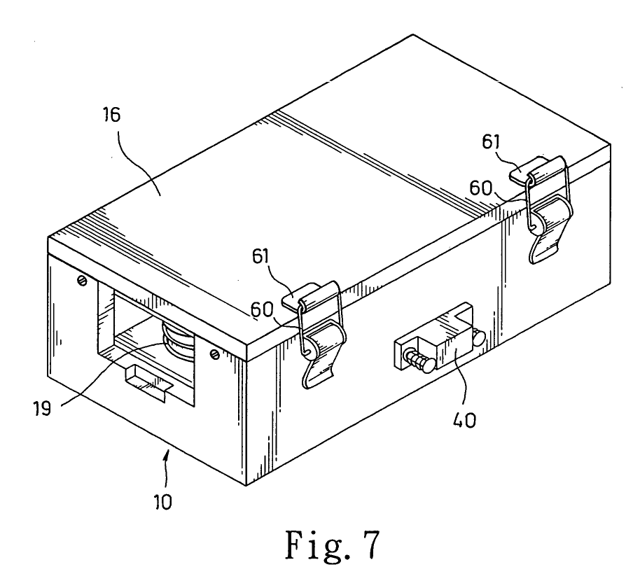

Referring further to FIG. 2, as shown in FIG. 2, the container 10 mainly includes a top-opened type box-shaped body and a cover 16 pivotally coupled to the box-shaped body for operably open/close the top opening of the box-shaped body, wherein the box-shaped body is consisted of a bottom 11 and four sidewalls 12, 13, 14 and 15. The top opening of the box-shaped body serves as a loading port of a bag containing medical fluid for placing/removing the bag containing medical fluid into/from the box-shaped body. An opening 17 and 18 is formed in the sidewall 12 and 14 respectively serving as a discharge port. There are four spring holding member 19 being provided on an upper face of the bottom 11 of the box-shaped body. In addition, the box-shaped body also includes two lock members 40 installed on the sidewalls 13 and 15 respectively. The lock member 40 extends through the sidewall into the interior of the box-shaped body and has a slant face at a distal end thereof. The lock member 40 is operably mounted to the sidewall 13 or 15 by two screws 43 wherein a return spring 42 is provided on each of the screw 43 such that the lock member 40 may be manually pulled outwardly along the screws 43 to extract the engaging tongue 41 into the sidewall 13 or 15 and may be pushed inward back to its original extended position by the return spring 42 when the lock member 40 is released. As shown in FIG. 2, the box-shaped further includes four stop members 50 mounted on sidewalls 12 and 14 respectively. The positions of the stop members 50 are selected such that when they are in contact with the push plate 20 and stop the upward movement of the push plate 20, the upper surface of the push plate 20 is flush with a top face of the sidewalls. In this embodiment, the stop members are four screws 50 and they can be screwed inside the box-shaped body to stop the push plate 20 or can be screwed out to facilitate the assembling/disassembling operation of the push plate 20. Moreover, the box-shaped body further includes two buckle members 60 for locking the cover 16 to the box-shaped body by engaging with the mating buckle members 61 provided on the cover 16 (FIG. 7) while the cover 16 closes the top opening of the box-shaped body.

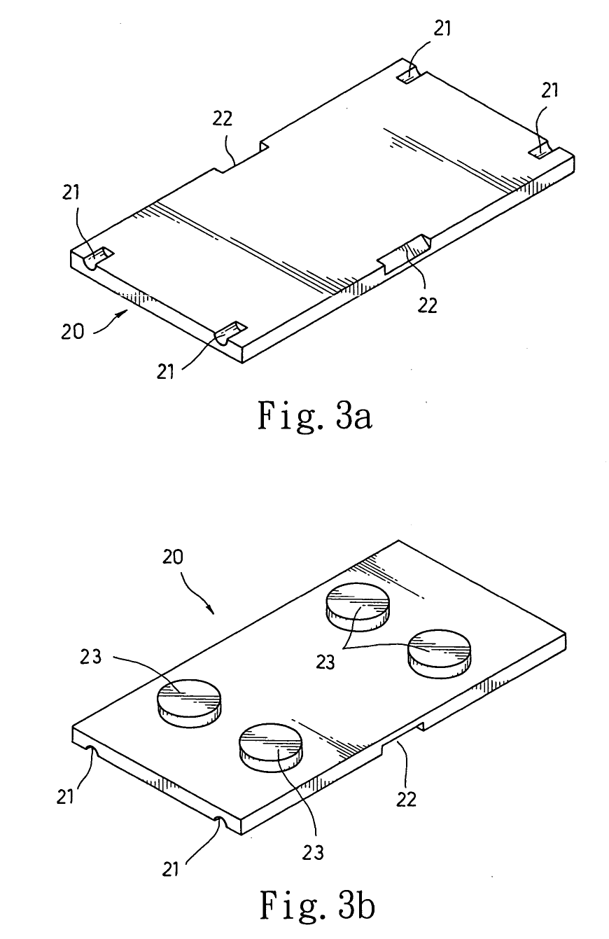

Referring now to FIGS. 3a and 3b to describe the structure of the push plate 20. As shown in the drawings, the push plate 20 has a shape substantially the same as that of the bottom 11 of the box-shaped body and a area slightly smaller than that of the bottom so that it can move smoothly in the box-shaped body. Four recesses 21 are formed in an upper surface of the push plate 20 at positions corresponding to the stop member 50 mounted on the sidewalls 12 and 14 and two recesses 22 are formed at positions corresponding to the lock members 40. In addition, four spring holding members 23 are provided on a lower surface of the push plate 20 at positions corresponding to the spring holding members 19 provided on the bottom 11 of the box-shaped body.

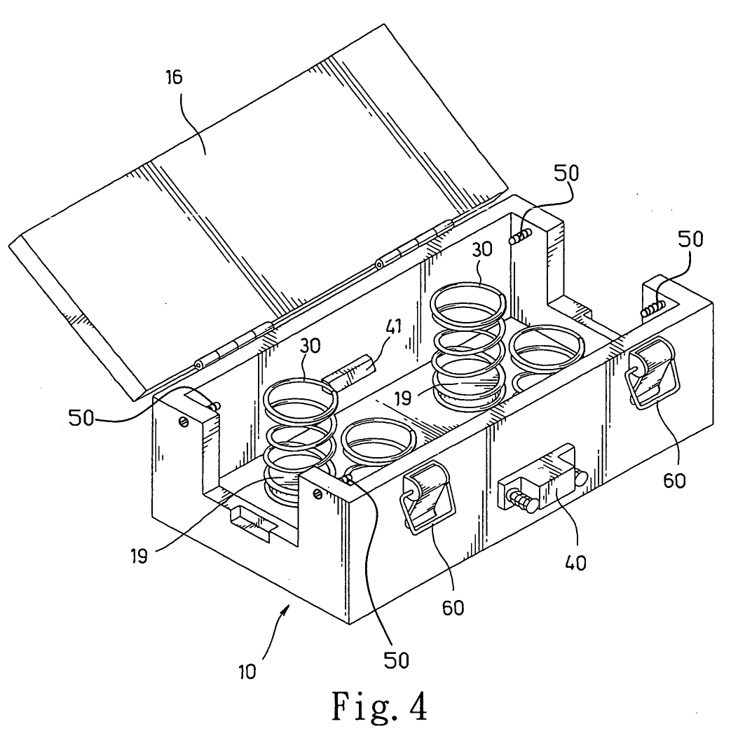

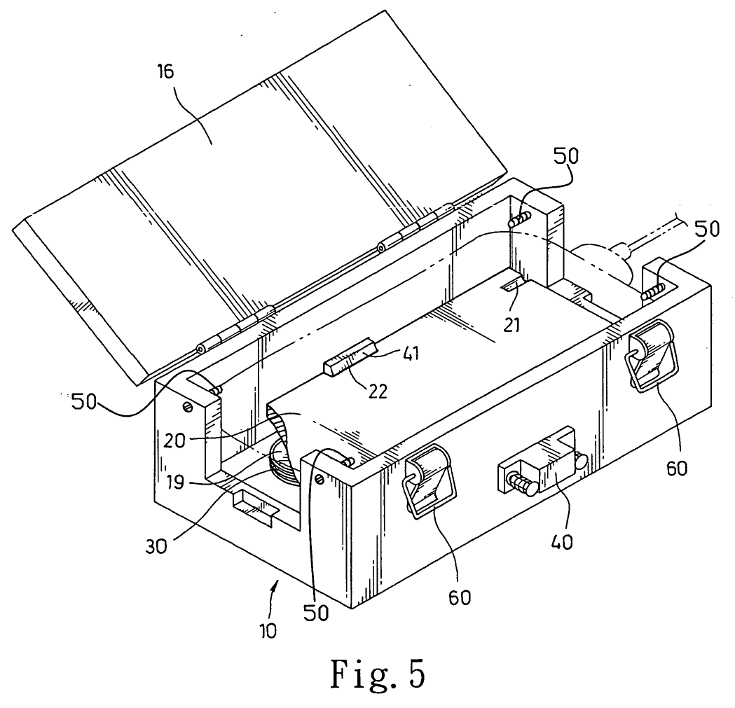

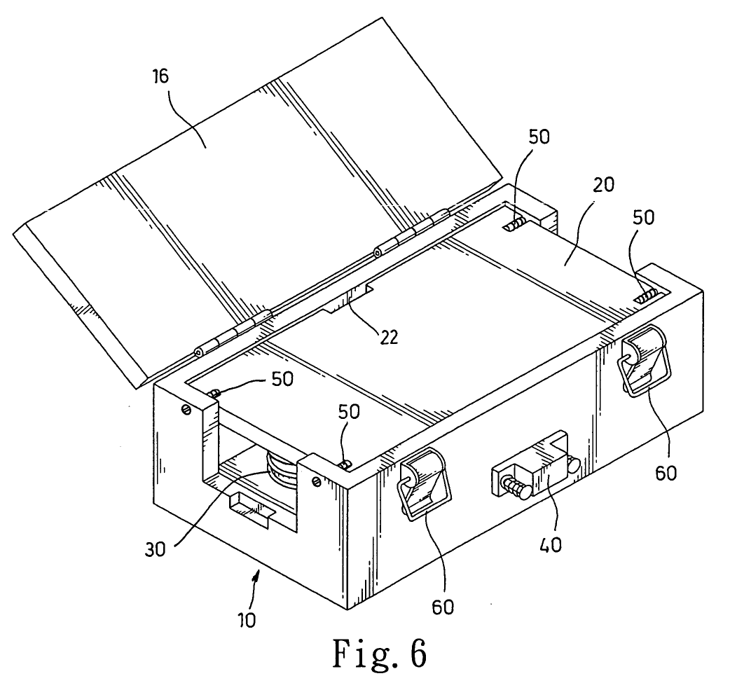

In assembly, the four spiral springs 30 are coupled to the spring holding members 19 provided on the bottom 11 of the box-shaped body, as shown in FIG. 4. Then, the stop members 50 are screwed backward so as to go into the sidewalls 12 and 14. Next, the push plate 20 is placed on top of the spiral springs 30 and coupled thereto by engaging the spring holding members 23 to the springs 30, and pushed downward until the push plate 20 is locked at the first position by the lock members 40 as shown in FIG. 5. Then, the stop members 50 are screwed forward so as to extend inside the box-shaped body again. When the push plate 20 is locked at the first position as illustrated in FIG. 5, a space is defined in the box-shaped body which is sufficient to accommodate a collapsible bag containing medical fluid (such as, a bag containing fluid for intravenous injection or a bag containing dialysis fluid). Then a bag containing medical fluid may be placed in the box-shaped body with a fluid outlet being disposed in one of the discharge ports 17 and 18 formed in the sidewall 12 and 14 (in this embodiment, the fluid outlet is disposed in discharge port 18 as shown in imagination lines of FIG. 5). Then, the cover 16 is placed on top of the box-shaped body to close the box-shaped body and is locked to the box-shaped body by engaging the lock members 61 provided on the cover 16 to the lock member 60 of the box-shaped body, as shown in FIG. 6.

When needs to discharge the fluid, the lock members 40 are pulled outwardly to disengage the engaging tongues 41 from the recesses 22. At this time, the push plate 20 is pushed upward by the spiral springs 30 and this spring force exerted to the push plate by the springs 30 is evenly passed on to the bag containing medical fluid to press the bag against the cover 16, and the medical fluid (such as, the fluid for intravenous injection or the dialysis fluid) is thus discharged out from the bag.

Since the stop members 50 and push plate 20 is designed such that the upper surface of the push plate 20 will be flush with the top face of the sidewalls while the push plate 20 is stopped by the stop members 50, the medical fluid contained in the bag can discharged completely. When the bag is emptied, the emptied bay can be removed by unlocking the lock members 60 and 61 and take away the cover 16. Then, the push plate 20 can be pushed down and locked at the first position by the lock member 40 once again to make the device ready for next use.

Because of the novel active pressuring design of the present invention as disclosed above, the portable device for transfusing medical fluid accommodated in a collapsible bag of the present invention can actively provide the pressure required to perform intravenous injection or dialysis fluid recharging. Thus, while performing the intravenous injection or dialysis fluid recharging, the bag containing medical fluid is no longer have to be hanged or placed at a position higher than the position performing the intravenous injection or dialysis fluid recharging. Instead, by placing the bag containing medical fluid in the portable device of the present invention, a intravenous injection or dialysis fluid recharging operation may be effectively and safely performed even if the portable device of the present invention which has a medical fluid bag accommodated therein is placed at a position mush lower than the patient. That is, performing the intravenous injection or dialysis fluid recharging is no longer limited to the hanging/positioning level of the bag containing medical fluid or dialysis fluid while using the device of the present invention. In addition, because the medical fluid transfusion device of the present invention is designed so compact that it can be put in a backpack and carried around very easily, the patient can thus live a life with a much higher quality by using the device of the present invention.

This invention has been disclosed in terms of specific embodiments. It will be apparent that many modifications can be made to the disclosed structures without departing from the invention. Therefore, it is the intent of the appended claims to cover all such variations and modifications as come within the sprit and scope of this invention.

What is claimed is:

1. A portable device for transfusing medical fluid accommodated in a collapsible bag comprising:
a container which includes a box-shaped body having a loading port for loading the bag of medical fluid and at least one fluid exit port, and a cove operably coupled to the box-shaped body for selective open/close the load port;
a push plate movably disposed in the container and able to be displaced between a first position and a second position, wherein when the push plate is at the first position a space is defined within the container for receiving the bag of medical fluid and when the push plate is at the second position the space is reduced to a degree that the medical fluid in the collapsible bag is substantially discharged;
a energy providing unit operably coupled to the push plate for providing energy required from displacing the push plate; and
a lock/unlock means mounted to the container for operably locking the push plate at the first position or release the push plate to allow it to move to the second position.

2. A portable device for transfusing medical fluid accommodated in a collapsible bag according to claim 1, wherein the energy providing unit comprises a set of springs which are consisted of at lest one spiral springs.

3. A portable device for transfusing medical fluid accommodated in a collapsible bag according to claim 1, wherein the box-shaped body further comprises a stopping means for stopping the push plate at the second position.

4. A portable device for transfusing medical fluid accommodated in a collapsible bag according to claim 2, wherein the box-shaped body is consisted of a bottom and four sidewalls and has a top opening which serves as a loading port for loading/unloading the collapsible bag.

5. A portable device for transfusing medical fluid accommodated in a collapsible bag according to claim 4, wherein at lest one first spring holding member is provided at the bottom of the box-shaped body for coupling one end of the at least one spiral spring and at lest one first spring holding member is provided at a lower face of the push plate for coupling the other end of the at least one spiral spring.

6. A portable device for transfusing medical fluid accommodated in a collapsible bag according to claim 4, wherein the stop means is designed such that an upper surface of the push plate will be flush with the top face of the sidewalls while the push plate is stopped at the second position by the stop means.

7. A portable device for transfusing medical fluid accommodated in a collapsible bag according to claim 1, wherein the box-shaped body further comprises a first lock member and the cover comprises a second lock member for engaging with the first lock member so as to lock the clove to the box-shaped body while the cover close the loading port of the box-shaped body.
